(19) United States
(12) Patent Application Publication
(10) Pub. No.: US 2019/0307802 A1
(43) Pub. Date: Oct. 10, 2019

(54) PROBIOTIC COMPOSITIONS INCLUDING IMMUNE MODULATORS

(71) Applicant: 4Life Patents, LLC, Sandy, UT (US)

(72) Inventors: David Vollmer, South Jordan, UT (US); Paula Brock, Salt Lake City, UT (US); Brent Vaughan, Draper, UT (US); Shane Lefler, American Fork, UT (US)

(21) Appl. No.: 16/380,952

(22) Filed: Apr. 10, 2019

Related U.S. Application Data
(60) Provisional application No. 62/655,742, filed on Apr. 10, 2018.

(51) Int. Cl. A61K 35/20; A61K 35/57; A61K 35/745; A61K 35/747; A61P 1/14; A23L 33/135; A23L 33/21

(52) U.S. Cl. CPC A61K 35/20; A23Y 2220/03; A61K 35/745; A61K 35/747; A61P 1/14; A23L 33/135; A23L 33/21; A23V 2002/00; A23V 2200/3202; A23V 2200/3204; A23V 2250/2044; A23V 2250/284; A23Y 2300/55; A23Y 2300/49; A23Y 2300/21; A23Y 2300/29; A23Y 2300/45; A23Y 2220/17; A23Y 2220/71; A23Y 2220/73; A61K 35/57

(57) ABSTRACT

A digestive product includes a probiotic component and an immune modulator, such as transfer factor and/or a nanofraction immune modulator. The digestive product may also contain a prebiotic component, such as one or more of galactooligosaccharides, xylooligosaccharides, and fructooligosaccharides. The probiotic component may be encapsulated, such as probiotic micro beadlets.

### CROSS-REFERENCE TO RELATED APPLICATION

A claim for the benefit of priority to the Apr. 10, 2019 filing date of U.S. Provisional Patent Application No. 62/655,742, titled DIGESTIVE PRODUCT (“the '742 Provisional application”) is hereby made pursuant to 35 U.S.C. § 119(e). The entire disclosure of the '742 Provisional application is hereby incorporated herein.

### TECHNICAL FIELD

This disclosure relates generally to compositions that aid digestion and, more specifically, to a probiotic composition that includes one or more probiotics, an immune modulator, and, optionally, one or more prebiotics. More specifically, the immune modulator of a probiotic composition according to this disclosure may comprise a nano-fraction immune modulator, transfer factor, or a combination of immune modulators. Methods for supporting a subject's digestion are also disclosed.

### SUMMARY

This disclosure includes various embodiments of probiotic compositions. A probiotic composition according to this disclosure may comprise, consist essentially of, or consist of a probiotic component and an immune modulating component. Alternatively, a probiotic composition of this disclosure may include, consist essentially or, or consist of a prebiotic component, a probiotic component, and an immune modulating component.

The probiotic component of a probiotic composition of this disclosure may include, consist essentially of, or consist of one or more types of probiotic bacteria, which are also more simply referred to as “probiotics.” Probiotics are live bacteria, or “microorganism” (MOs) that, when consumed by a subject, are intended to restore or improve the normal flora that live in the subject's gut (i.e., gastrointestinal tract), thereby benefiting the subject's digestive health, as well as his or her overall health. A probiotic component that consists essentially of one or more probiotics may also include substances that support and/or stabilize the probiotic(s) during its (their) growth and/or storage.

The immune modulating component of a probiotic composition according to this disclosure may comprise or consist essentially of one or more immune modulators. Examples of immune modulators include, without limitation, transfer factor, nanofraction immune modulators, and combinations of transfer factor and nanofraction immune modulators. An immune modulating component that consists essentially of one or more immune modulators may include the immune modulator, as well as other molecules from a source (e.g., colostrum, egg, etc.) from which each immune modulator was obtained. Each immune modulator of the immune modulating component of the probiotic composition may retain or substantially retain one or more of its activities (e.g., its ability to regulate, or modulate, a subject's immune system; its ability to enhance the effectiveness of antioxidants in the subject's body; its ability to achieve and maintain oxidative balance in the subject's body; etc.) when present in the probiotic composition; i.e., the immune modulator may function without interference from other components of the probiotic composition.

Various embodiments of probiotic compositions of this disclosure may include a prebiotic component. The prebiotic component may comprise, consist of, or consist essentially of one or more saccharides. The prebiotic component may facilitate, or support, growth of one or more probiotics of the probiotic component.

A probiotic composition according to this disclosure may be in a solid form. Without limitation, the probiotic composition may comprise a powder (e.g., in individual, pre-measured satchets, etc.), a capsule, a tablet, a caplet or any other pre-measured oral dosage form. Alternatively, a probiotic composition may comprise a liquid, or it may be provided in any other suitable form.

Methods for supporting a subject's gut and/or the subject's digestion may include administering a probiotic composition that includes, consists essentially of, or consists of a probiotic component, an immune modulating component, and an optional prebiotic component.

Other aspects of the disclosed subject matter, as well as features and advantages of various aspects of the disclosed subject matter, will become apparent to those of ordinary skill in the art through consideration of the ensuing description and the appended claims.

### BRIEF DESCRIPTION OF THE DRAWINGS

In the drawings:

FIG. 1 illustrates an embodiment of a test plate used to determine the effects of various prebiotics and an immune modulator on various probiotics;

FIG. 2 depicts the contents of each well of the test plate shown in FIG. 1;

FIGS. 3-6 are charts comparing the effects of an embodiment of nanofraction immune modulator to the effects of various combinations of prebiotics on the growth and/or proliferation of various probiotics;

FIG. 7 is a chart illustrating the design for a study for evaluating the effects of an embodiment of a probiotic composition according to this disclosure on mice;

FIG. 8 is a chart showing the results of a short chain fatty acid (SCFA) analysis conducted in the study of FIG. 7;

FIG. 9 depicts the phylum level taxonomy of the gut microbiome of the mice used in the study of FIG. 7;

FIG. 10 depicts the family level taxonomy of the gut microbiome of the mice used in the study of FIG. 7;

FIG. 11 is a chart depicting the alpha diversity of the gut microbiome, as determined in the study of FIG. 7;

FIG. 12 is a spatial representation of beta diversity of the gut microbiome, as determined by Principal Coordinates Analysis (PCoA) in the study of FIG. 7;

FIG. 13 is a spatial representation of the difference in beta diversities observed between a control group of mice and a group of mice that received the embodiment of prebiotic composition in the study of FIG. 7;

FIG. 14 is a chart showing the effects of the various diets administered in the study of FIG. 7 on levels of fecal calprotectin; and

FIG. 15 is a chart showing the effects of the various diets administered in the study of FIG. 7 on levels of plasma zonulin.

### DETAILED DESCRIPTION

In various embodiments, a probiotic composition according to this disclosure may include a combination of a probiotic component and an immune modulating component. Additionally, a probiotic composition according to this disclosure may include a prebiotic component.

The probiotic component of the probiotic composition may include one or more probiotics. Examples of probiotics that may be included in the probiotic component include, but are not limited to, Bifidobacterium longum subsp. longum (e.g., BB536-ATCC BAA-999) (hereinafter “B. longum”), Bifidobacterium animalis subsp. lactis (e.g., Bl-04-ATCC SD-5219) (hereinafter “B. lactis”), B. animalis, Bifidobacterium longum subsp infantis (e.g., M-63-BCCM LMG 23728) (hereinafter “B. infantis”), B. breve, Lactobacillus casei, L. reuteri, L. rhamnosus (e.g., L. rhamnosus GG (Lr-32)-ATCC SD-5217), and L. acidophilus (e.g., L. acidophilus (NCFM)-ATCC SD-5221). In a specific embodiment, the probiotic component may comprise, consist essentially of, or consist of one or more of B. longum, B. lactis, B. animalis, B. infantis, B. breve, L. casei, L. reuteri, L. rhamnosus, L. acidophilus.

In some embodiments, the probiotic (alone or in combination with one or more other components of the digestive product) may have a form that enables it to retain its viability (e.g., percentage of living cells, etc.) or activity or substantially retain its viability or activity (e.g., retain at least 75% of its viability or activity, retain at least 50% of its viability or activity, retain at least 25% of its viability or activity, retain at least 10% of its viability or activity, etc.) as it travels through an individual's digestive tract to his or her gut. For example, the probiotic (alone or in combination with one or more other components of the digestive product) may be encapsulated, which may improve delivery of the probiotic to the gut of a subject. As an example, the probiotic component of a probiotic composition according to this disclosure may comprise, consist essentially of, or consist of a Jintan Probiotic 8B CFU (colony forming units) beadlet (Morishita Jintan Co., Ltd., Osaka, Japan).

The immune modulating component of the probiotic composition may include one or more types of immune modulators. As an example, the immune modulating component may comprise transfer factor. The transfer factor may be obtained from any suitable, acceptable source. Without limitation, the transfer factor may be obtained from colostrum (e.g., bovine colostrum, etc.), as disclosed by U.S. Pat. No. 4,816,563 to Wilson et al. (hereinafter “Wilson”), the entire disclosure of which is hereby incorporated herein. The transfer factor may be obtained from eggs (e.g., chicken eggs, etc.), as disclosed by U.S. Pat. No. 6,468,534 to Hennen et al. (hereinafter “Hennen”), the entire disclosure of which is hereby incorporated herein. The immune modulating component may include a combination of two or more types of transfer factor, as disclosed by U.S. Pat. No. 6,866,868 to Lisonbee et al. (hereinafter “Lisonbee Bovine-Avian TF”), the entire disclosure of which is hereby incorporated herein. Transfer factor modulates, or regulates, the immune system of a subject (e.g., cell-mediated immunity, etc.). Transfer factor also enhances the effectiveness of antioxidants in a subject's body and improves oxidative balance in a subject's body, as demonstrated by the international patent application filed pursuant to the Patent Cooperation Treaty and having International Publication Number WO 2004/041071 A2 (hereinafter “Dadali”), the entire disclosure of which is hereby incorporated herein.

As an alternative to transfer factor, or in addition to transfer factor, the immune modulating component of a probiotic composition according to this disclosure may include a nanofraction immune modulator of the type disclosed by Nanofraction immune modulators are also disclosed by US 2008/0081076 A1 of Lisonbee et al. (hereinafter “Lisonbee NanoFactor/Tri-Factor”), the entire disclosure of which is hereby incorporated herein. A nanofraction immune modulator may have an upper molecular weight cutoff that excludes transfer factor (e.g., about 3,000 Da, about 4,000 Da, etc.).

Lisonbee NanoFactor/Tri-Factor also discloses immune modulating components that include transfer factor and nanofraction immune modulators. More specifically, Lisonbee NanoFactor/Tri-Factor discloses an immune modulating component that includes a fraction of colostrum that includes nanofraction immune modulators, a fraction of colostrum that includes transfer factor and nanofraction immune modulators, and a component obtained from chicken eggs.

The immune modulating component may be mixed or blended with (e.g., dispersed throughout a powder, dissolved in a liquid, etc.) the probiotic component.

Immune modulators obtained from bovine colostrum may include one or more saccharides, which may act as prebiotics, which are substances that promote the growth and proliferation of probiotics. Without limitation, an immune modulating component that includes one or more immune modulators derived from bovine colostrum may include (non-essentially in embodiments of the probiotic composition where prebiotics are not a required component; non-essentially or essentially in embodiments of the probiotic composition where a prebiotic component is required) 3′-sialyllactose, 6′-sialyllactose, and 6′-sialyl-N-acetyllactosamine.

In addition to any saccharides that accompany the immune modulating component, the probiotic composition may include a prebiotic component. The prebiotic component may comprise one or more saccharides. Saccharides may be chosen based on their effect on probiotic growth. Examples of saccharides that may be used in the prebiotic component include, without limitation, inulin, fructans, transgalactooligosaccharides. The saccharide may comprise one or more of a galactooligosaccharide (GOS), a xylooligosaccharide (XOS), and a fructooligosaccharide (FOS), etc. In a specific embodiment, the prebiotic component of a probiotic composition may comprise, consist essentially of, or consist of GOS, FOS, and/or XOS. When a combination of saccharides is used, the saccharides may be used in any suitable ratio. For example, a ratio of 2:1:2 of XOS:FOS:GOS may be used as the prebiotic component of a probiotic composition according to this disclosure.

Probiotic compositions according to this disclosure may be provided in a solid oral dosage form. Without limitation, a probiotic composition may be provided in the form of a powder (e.g., in individual satchets that contain a premeasured amount of the probiotic composition to be administered orally or mixed into water or another liquid, etc.), in capsules, in tablets, or in any other solid form that provides a premeasured quantity that corresponds to a part or all of a recommended daily dose. Alternatively, a probiotic composition according to this disclosure may be provided in a liquid form (e.g., an orally administrable composition, as part of a drink (e.g., a fruit drink, etc.), etc.).

A specific embodiment of a probiotic composition according to this disclosure is set forth in TABLE 1.

TABLE 1

| Component | Quantity | |
|---|---|---|
| Jintan Probiotic beadlet 8 B CFU/ | 250.0000 mg | (8.065%) |
| g(Hydrogenated Oil, Fish gelatin, | | |
| glycerin, soy lecithin, pectin | | |
| Galactooligosaccharides (GOS) | 1,000.0000 mg | (32.258%) |
| Xylooligosaccharides (XOS) | 1,000.0000 mg | (32.258%) |
| Fructooligosaccharides (FOS) | 500.0000 mg | (16.129%) |
| Dried Egg Yolk Powder (Avian) | 30.0000 mg | (0.969%) |
| Colostrum Filtrate | 68.6000 mg | (2.216%) |
| Colostrum Extract (NanoFactor ®, 4Life | 1.4000 mg | (0.044%) |
| Research, LLC, Sandy, Utah; see, e.g., | | |
| Lisonbee NanoFactor/Tri-Factor | | |
| Maltodextrin | 105.0000 mg | (3.387%) |
| Lemon flavor | 23.2300 mg | (0.749%) |
| Stevia [NLT 80% Steviol glycosides] | 1.3700 mg | (0.044%) |
| Cane sugar | 110.4000 mg | (3.561%) |
| Salt (Sodium Chloride) | 10.0000 mg | (0.323%) |

TABLE 2 shows the active ingredients of the probiotic composition of TABLE 1.

TABLE 2

| | Amount per unit/ | | Amount per group/ | | Amount of probiotics/ | |
|---|---|---|---|---|---|---|
| Active Ingredients | daily serving | | daily serving | | daily serving | |
| Jintan Probiotic beadlet 8 B CFU: | | | | | | |
| Bifidobacterium longum subsp | 500 | million | 1.5 | billion | 2 | billion |
| infantis. (M-63) - BCCM LMG | (5E8) | CFU | (1.5E9) | CFU | (2.0E9) | CFU |
| 23728 | | | | | | |
| Bifidobacterium longum subsp. | 500 | million | | | | |
| longum (BB536) - ATCC BAA- | (5E8) | CFU | | | | |
| 999 | | | | | | |
| Bifdobacterium animalis subsp. | 500 | million | | | | |
| lactis (Bl-04) - ATCC SD-5219 | (5E8) | CFU | | | | |
| Lactobacillus rhamnosus GG (Lr- | 250 | million | 0.5 | billion | | |
| 32) - ATCC SD-5217 | (2.5E8) | CFU | (0.5E9) | CFU | | |
| Lactobacillus acidophilus (NCFM) - | 250 | million | | | | |
| ATCC SD-5221 | (2.5E8) | CFU | | | | |
| Xylooligosaccharides (XOS) | 1000 | mg | 2500 | mg | | |
| Galactooligosaccharides (GOS) | 1000 | mg | (2.5 | g) | | |
| Fructooligosaccharides (FOS) | 500 | mg | | | | |
| Dried Egg Yolk Powder (Avian) | 30.0000 | mg | | | | |
| Colostrum Filtrate | 68.6000 | mg | | | | |
| Colostrum Extract (NanoFactor, 4Life | 1.4000 | mg | | | | |
| Research, LLC, Sandy, Utah; see, e.g., | | | | | | |
| Lisonbee NanoFactor/Tri-Factor | | | | | | |

A daily dosage of about 3.1 grams of a composition with ingredients in the proportions listed in TABLE 1 may be administered to or consumed by a subject. It may be beneficial to take the daily dosage of about 3.1 grams daily for about thirty (30) days, and then take the daily dosage of about 3.1 grams every other day.

Known benefits of probiotics and prebiotics include, without limitation: increase in prevalence and/or growth of health-promoting microorganisms within the colon and intestines (Lahtinen, S., et al., “Probiotic cheese containing Lactobacillus rhamnosus HN001 and Lactobacillus acidophilus NCFM(R) modifies subpopulations of fecal lactobacilli and Clostridium difficile in the elderly,” Age (Dordr) 34(1):133-143 (2012) (hereinafter “Lahtininen”); reduction in the prevalence of unwanted bacteria within the gut (Lahtinen); support for healthy immune system function (Ibrahim, F., et al., “Probiotics and immunosenescence: cheese as a carrier,” FEMS Immunol. Med. Microbiol. 59(1):53-59 (2010)); support for bowel function and overall bowel health (Pregliasco, F., et al., “A new chance of preventing winter diseases by the administration of synbiotic formulations,” J. Clin. Gastroenterol. 42 Suppl. 3 Pt. 2:S224-233 (2008)); and support healthy cholesterol levels and insulin function (Andrade, S., “Effect of fermented milk containing Lactobacillus acidophilus and Bifidobacterium longum on plasma lipids of women with normal or moderately elevated cholesterol,” J. Dairy Res. 76(4):469-474 (2009)); Vulevic, J., et al., “A mixture of trans-galactooligosaccharides reduces markers of metabolic syndrome and modulates the fecal microbiota and immune function of overweight adults,” J. Nutr. 143(3): 324-331 (2013); Chonan, O., et al., “Effect of galactooligosaccharides on calcium absorption and preventing bone loss in ovariectomized rats,” Biosci. Biotechnol. Biochem. 59(2):236-239 (1995)).

In addition to the numerous known and believed benefits of probiotics and prebiotics, administration or consumption of an immune modulating component along with a probiotic composition or as part of the probiotic composition provides the subject with additional and even synergistic beneficial effects. While the disclosures of Wilson, Hennen, Lisonbee Bovine-Avian TF, Dadali, and Lisonbee NanoFactor/Tri-Factor note the benefits of immune modulators in humans and other mammals, they do not discuss the benefits of immune modulators, such as transfer factors, nanofraction immune modulators, and the like, on prokaryotic organisms, such as probiotic organisms. The effects of immune modulating components on probiotic organisms (e.g., the rate at which probiotic microorganisms multiply when grown in the presence of immune modulating components, their activities, etc.), as well as the benefits immune modulating components provide when combined with probiotics and, optionally, with prebiotics, were previously unknown and somewhat unexpected.

### Example 1

The effects of various combinations of prebiotics and the effects of immune modulators on the proliferation of five (5) different types of probiotics (L. rhamnosus, L. acidophilus, B. longum, B. infantis, and B. lactis) were evaluated. B. infantis, B. longum, and B. lactis powdered cultures were inoculated, using a sterile swab, into tubes containing 9 mL of a 20% MRS (De Man, Rogosa, and Sharpe) broth (which broth concentration was preselected based on its ability to stimulate growth when incubated with B. lactis and L. acidophilus when incubated at 37° C. for 12 hrs.) supplemented with 0.05% L-cysteine, placed in an anaerobic container (GasPak, Becton, Dickinson and Company, Franklin Lakes, N.J.) and then incubated for 24 hrs. at 37° C. L. rhamnosus and L. acidophilus cultures were inoculated into tubes containing 9 mL of a 20% MRS broth and incubated for 24 hrs. at 37° C. Following incubation, each culture was transferred to a sterile tube and back diluted with a 20% MRS broth supplemented with 0.05% L-cysteine to an OD₆₀₀ of 0.1.

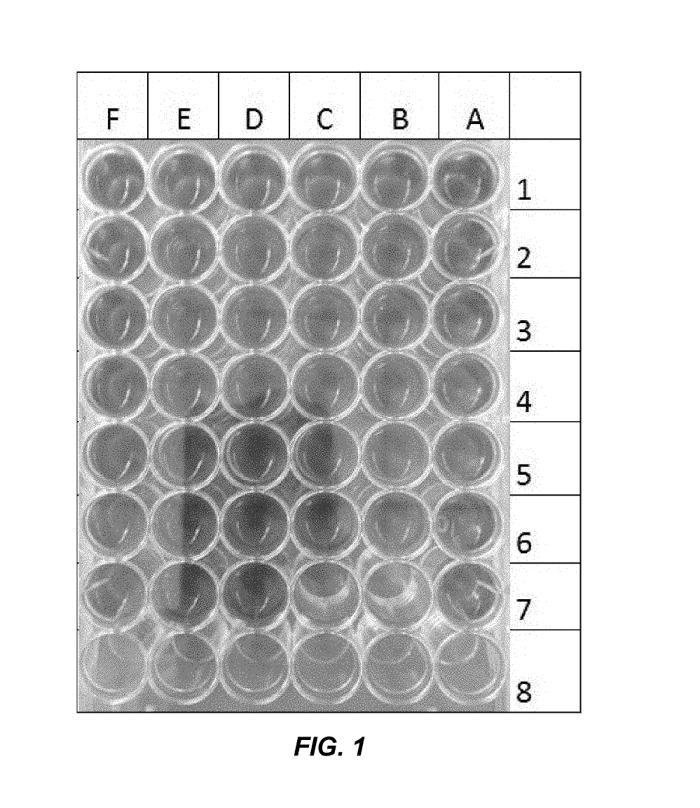

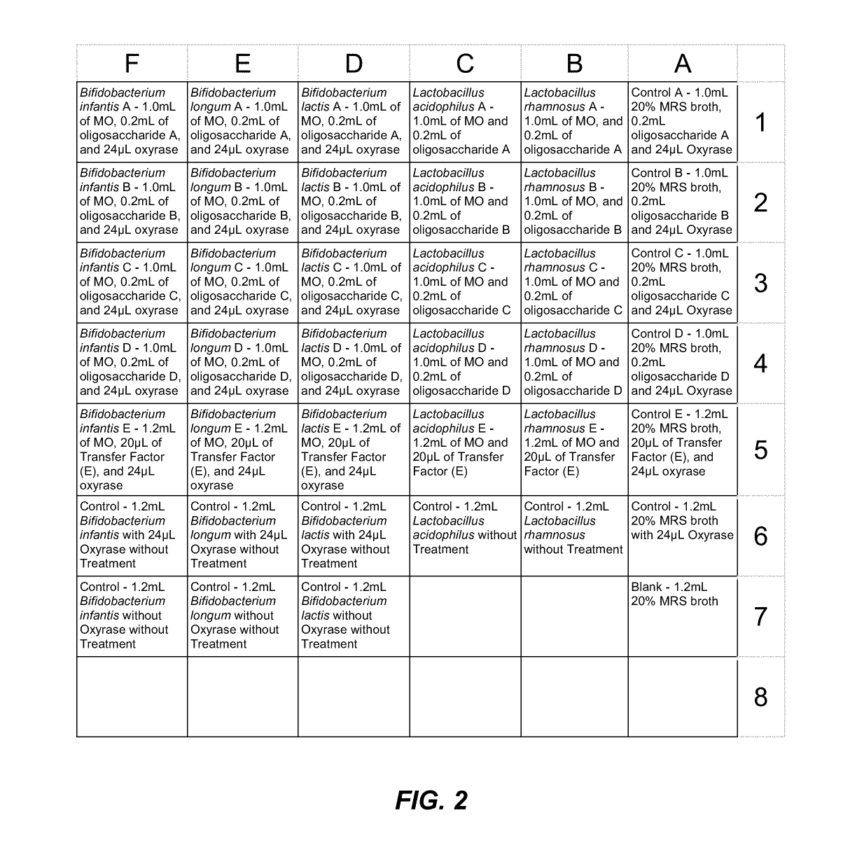

One milliliter (1 mL) aliquots of each diluted organism (0.1 at OD₆₀₀) were then added to selected wells of a 48-well microtiter plate, as illustrated by FIG. 1 and as specified by FIG. 2. More specifically, as specified by FIG. 2: 1 mL aliquots of B. longum were added to each of four designated wells (rows 1-4) of a first column (column F) of the 48-well microtiter plate and 1.2 mL aliquots of B. longum were added to each of three more designated wells (rows 5-7) of the first column (column F) of the 48-well microtiter plate; 1 mL aliquots of B. infantis were added to each of four designated wells (rows 1-4) of a second column (column E) of the 48-well microtiter plate and 1.2 mL aliquots of B. infantis were added to each of three more designated wells (rows 5-7) of the second column (column E) of the 48-well microtiter plate; 1 mL aliquots of B. lactis were added to each of four designated wells (rows 1-4) of a third column (column D) of the 48-well microtiter plate and 1.2 mL aliquots of B. lactis were added to each of three more designated wells (rows 5-7) of the third column (column D) of the 48-well microtiter plate; 1 mL aliquots of L. rhamnosus were added to each of four designated wells (rows 1-4) of a fourth column (column C) of the 48-well microtiter plate and 1.2 mL aliquots of L. rhamnosus were added to each of two more designated wells (rows 5 and 6) of the fourth column (column C) of the 48-well microtiter plate; and 1 mL aliquots of L. acidophilus were added to each of four designated wells (rows 1-4) of a fifth column (column B) of the 48-well microtiter plate and 1.2 mL aliquots of L. acidophilus were added to each of two more designated wells (rows 5 and 6) of the fifth column (column B) of the 48-well microtiter plate. Five of the wells that contained each probiotic (the four 1 mL samples (in rows 1-4) and the first 1.2 mL sample (in row 5)) were designated for different treatments, while the two remaining wells (in rows 6 and 7) that contained 1.2 mL of each of B. longum, B. infantis, and B. lactis and the one remaining well (in row 6) that contained 1.2 ml of each of L. rhamnosus and L. acidophilus were designated as controls, as set forth in FIG. 2.

As probiotic-free controls, each of the first four wells (rows 1-4) of a sixth column (column A) of the 48-well microtiter plate received 1 mL of a 20% MRS broth supplemented with 0.05% L-cysteine, while the next three wells (rows 5-7) of the sixth column (column A) received 1.2 mL of a 20% MRS broth supplemented with 0.05% L-cysteine.

Five different treatments were also prepared, including four prebiotic treatments with XOS, FOS, and GOS in various ratios and an immune modulator-based treatment. The four prebiotic treatments included: Treatment A=2:2:1 XOS:FOS:GOS; Treatment B=1:1:1 XOS:FOS:GOS; Treatment C=1:2:2 XOS:FOS:GOS; and Treatment D=2:1:2 XOS:FOS:GOS. The immune modulator-based treatment, Treatment E, included Tri-Factor® immune modulator (4Life Research, LLC, Sandy, Utah), which includes a bovine colostrum fraction, dried egg yolk powder, and nano-filtered bovine colostrum in a 68:30:2 weight ratio. See Lisonbee NanoFactor/Tri-Factor. Each treatment was provided in solid, powdered form, with 2.5 g of each powder being introduced into a sterile tube containing 25 mL of sterile distilled water.

As further specified by FIG. 2, a 0.2 mL sample of Treatment A was added to a first well (in row 1) for each probiotic (columns F-B) and the probiotic-free controls (column A), a 0.2 mL sample of Treatment B was added to a second well (in row 2) for each probiotic (columns F-B) and the probiotic-free controls (column A), a 0.2 mL sample of Treatment C was added to a third well (in row 3) for each probiotic (columns F-B) and the probiotic-free controls (column A), and a 0.2 mL sample of Treatment D was added to a fourth well (in row 4) for each probiotic (columns F-B) and the probiotic-free controls (column A). The fifth well (in row 5) for each probiotic (columns F-B) and the fifth well (in row 5) for the probiotic-free controls (column A) received 20 μL of Treatment E. The reduced volume for Treatment E relative to the volumes of Treatments A-D ensured that the plate reader could correctly read the absorbance of the contents of the wells (in row 5) that contained the immune modulator.

To provide an anaerobic environment in certain wells, 24 μL of oxyrase (equating to about two percent (2%) of the volume in 1.2 mL of solution) was added to each of the first six samples (rows 1-6) of Bifidobacterium (in columns F, E, and D) and to the first six probiotic-free control samples (column A, rows 1-6). No oxyrase was added to any of the Lactobacillus samples (columns C and B, rows 1-6), to the final row (row 7) of Bifidobacterium samples, or to the final row (row 7) of probiotic free control samples (column A). Notably, one of the controls (column A, row 6) was probiotic-free and treatment-free, but included oxyrase, while another of the controls (column A, row 7) was probiotic-free, treatment-free, and oxyrase-free (i.e., it only included the 20% MRS broth supplemented with 0.05% L-cysteine).

The process was conducted in triplicate (i.e., three (3) 48-well microtiter plates were prepared.

The 48-well microtiter plates were then incubated at 37° C. for 12 hrs. The absorbance of each well was determined every 30 minutes during incubation. TABLE 3 shows the results of the growth of the probiotic cultures. (+) indicates increased growth relative to the respective probiotic-containing control with treatment, (−) indicates no difference in growth with treatment vs. the respective probiotic-containing control, (+/−) indicates some enhancement in growth with treatment relative to the respective probiotic control, which lacked any treatment.

TABLE 3

| Bacteria | Trial | Trt. A | Trt. B | Trt. C | Trt. D | Trt. E |
|---|---|---|---|---|---|---|
| Lb. rhamnosis | R4 | − | − | − | − | − |
| | R5 | − | − | − | − | − |
| | R6 | − | − | − | − | − |
| Lb. acidophilus | R4 | − | +/− | +/− | +/− | + |
| | R5 | − | +/− | +/− | +/− | + |
| | R6 | − | +/− | +/− | +/− | + |
| B. longum | R4 | − | − | − | − | − |
| | R5 | +/− | +/− | +/− | + | + |
| | R6 | +/− | +/− | +/− | + | + |
| B. infantis | R4 | − | − | − | − | − |
| | R5 | − | − | − | − | +/− |
| | R6 | − | − | − | − | + |
| B. lactis | R4 | +/− | + | + | + | + |
| | R5 | +/− | + | + | + | + |
| | R6 | +/− | +/− | +/− | +/− | + |

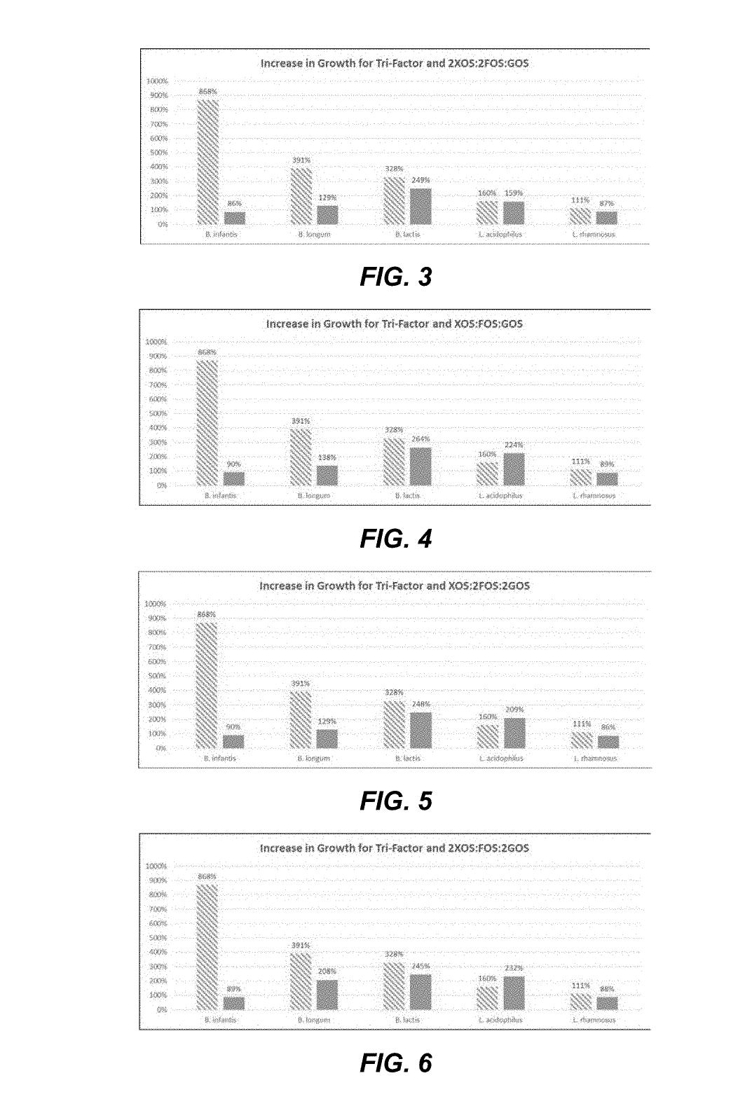

Treatment E (the Tri-Factor® immune modulator) increased the growth of each probiotic over its respective probiotic-containing control. In nearly every case, the extent to which Treatment E increased the growth of each probiotic exceeded the extent to which each of Treatments A, B, C, and D increased growth of that probiotic. FIGS. 3-6 show the effect of Treatment E on growth of the five (5) probiotics that were evaluated relative to the effects of each of Treatments A, B, C, and D, respectively, on the growth of each of those probiotics. In each of FIGS. 3-6, the effect of Treatment E on growth of the probiotics is indicated by the bars with diagonal lines, while the effect of the respective comparative treatment (Treatment A in FIG. 3, Treatment B in FIG. 4, Treatment C in FIG. 5, treatment D in FIG. 6) is indicated by the bars that are solid.

FIG. 3 shows the increase in growth for Treatment E vs. the increase in growth for Treatment A (2XOS:2FOS:1GOS) after 10 hours. For B. infantis, Treatment E caused an 868% increase in growth compared to 86% for Treatment A. For B. longum, Treatment E caused a 391% increase in growth compared to 129% for Treatment A. For B. lactis, Treatment E caused a 328% increase in growth compared to 249% for Treatment A. For L. acidophilus, Treatment E caused a 160% increase in growth compared to 159% for Treatment A. For L. rhamnosus, Treatment E caused a 111% increase in growth compared to 87% for Treatment A.

FIG. 4 shows the increase in growth for Treatment E compared to the increase in growth for Treatment B (XOS:FOS:GOS) after 10 hours. For B. infantis, Treatment E caused an 868% increase in growth compared to 90% for Treatment B. For B. longum, Treatment E caused a 391% increase in growth compared to 138% for Treatment B. For B. lactis, Treatment E caused a 328% increase in growth compared to 264% for Treatment B. For L. acidophilus, Treatment E caused a 160% increase in growth compared to 224% for Treatment B. For L. rhamnosus, Treatment E caused a 111% increase in growth compared to 89% for Treatment B.

FIG. 5 shows the increase in growth for Treatment E compared to the increase in growth for Treatment C (XOS:2FOS:2GOS) after 10 hours. For B. infantis, Treatment E caused an 868% increase in growth compared to 90% for Treatment C. For B. longum, Treatment E caused a 391% increase in growth compared to 129% for Treatment C. For B. lactis, Treatment E caused a 328% increase in growth compared to 248% for Treatment C. For L. acidophilus, Treatment E caused a 160% increase in growth compared to 209% for Treatment B. For L. rhamnosus, Treatment E caused a 111% increase in growth compared to 86% for Treatment C.

FIG. 6 shows the increase in growth of Treatment E compared to the increase in growth for Treatment D (2XOS:FOS:2GOS) after 10 hours. For B. infantis, Treatment E caused an 868% increase in growth compared to 89% for Treatment D. For B. longum, Treatment E caused a 391% increase in growth compared to 208% for Treatment D. For B. lactis, Treatment E caused a 328% increase in growth compared to 245% for Treatment D. For L. acidophilus, Treatment E caused a 160% increase in growth compared to 232% for Treatment D. For L. rhamnosus, Treatment E caused a 111% increase in growth compared to 88% for Treatment D.

### Example 2

Another study was conducted to evaluate the effect of a human gut health supplement in a mouse model when provided at a physiologically relevant dose. As a control, mice were fed the Total Western Diet (TWD), a purified rodent diet that matches the average U.S. intake of macronutrients and micronutrients [38]. The TWD was supplemented with prebiotics, probiotics, or immune modulators (a combination of a bovine colostrum filtrate, a bovine colostrum extract, and dried chicken egg yolk) individually and in combination. The endpoints of interest were the effect on the composition of the gut microbiome, cecal and fecal short chain fatty acids, gut inflammation, and plasma zonulin.

### Method and Materials

### Diet Formulation

Treatment dosages were calculated using a nutrient density approach to convert the dosage of an embodiment of probiotic composition according to this disclosure (Pre/o Biotics®, 4Life, Sandy, Utah—TABLE 1) to metabolically equivalent doses in mice, as set forth in TABLE 4.

TABLE 4
Translation of human to mouse intakes using nutrient density

| | | | | | | Immune | |
|---|---|---|---|---|---|---|---|
| Nutrient intake | | Prebiotics | | Probiotics | | Modulators | |
| Human | Pre/o Biotics supplement | 2.5 | g/d | 2 × 10⁹ | CFU/d | 100 | mg/d |
| | Energy intake(kcal/d) | 2500 | | 2500 | | 2500 | |
| | Nutrient density | 1 | mg/kcal | 8 × 10⁵ | CFU/kcal | 40 | μg/kcal |
| Mice | Translated dose | 11 | mg/d | 8.8 × 10⁶ | CFU/kcal | 0.44 | mg/d |
| | Energy intake (kcal/d) | 11 | | 11 | | 11 | |
| | Nutrient density | 1 | mg/kcal | 8 × 10⁵ | CFU/kcal | 40 | μg/kcal |
| | Actual dose^{§} | 16.5 | mg/d | 1.3 × 10⁷ | CFU/d | 0.66 | mg/kcal |

^{§}The prebiotic and immune modulator were increased by 1.5-fold and the probiotics by 3 fold to increase likelihood of measurable effects

The probiotic composition, Pre/o Biotics, includes 2.5 g of prebiotics with equal parts fructooligosaccharides (FOS), galactooligosaccharides (GOS) and xylooligosaccharides (XOS). In addition, the probiotic composition includes 0.5×10⁹ CFU of each of B. infantis (M-63), B. longum (BB536) and B. lactis (Bl-04), and 0.25×10⁹ CFU of each of L. rhamnosus (Lr-32) and L. acidophilus (NCFM). The probiotic composition also includes 100 mg of an immune modulating component comprising a proprietary concentrate of egg yolk and bovine colostrum proteins and peptides.

To convert the dosages using nutrient density, an average caloric intake of 2,500 kcal day was used for humans. For mice, 11 kcal was determined using a number or previous studies in our group. The quantities of prebiotics, probiotics, and immune modulator in the probiotic composition were normalized to an average human caloric intake (i.e. 2.5 g prebiotics/2500 kcal=1 mg/kcal). This value was then used to determine the mass added to the TWD formulation, which has a 4400 kcal per kilogram. For the prebiotics, there should be 4.4 g of prebiotics per kg of diet (i.e. 1 mg/kcal*4400 kcal), and similar calculations were made for the probiotics and immune modulators. To increase the likelihood of measuring treatment effects, the doses of prebiotics and immune modulators were increased 1.5-fold, and the probiotic treatment 3-fold (TABLE 4).

The control diet was the Total Western Diet (TWD) and, for the treatment groups, a portion of maltodextrin was removed to account for the addition of the probiotic composition. The decision to replace maltodextrin was made as it has most often been used as a control in human prebiotic studies [39, 40]. Diet assignments were as follows: 1) TWD: Total Western Diet as control: 2) PRE: prebiotics, 3) PRO: probiotics, 4) TF: immune modulators (Tri-Factor®, 4Life Research, LLC, Sandy, Utah), 5) COM: prebiotics, probiotics, and immune modulators (Pre/o Biotics®, 4Life Research, LLC, Sandy, Utah). The composition of the diets is shown in TABLE 5.

TABLE 5
Composition of experimental diets

| | | | | | |
|---|---|---|---|---|---|
| Diet composition | TWD | PRE | PRO | TF | COM |

Treatment (g/kg)

| | | | | | |
|---|---|---|---|---|---|
| Prebiotic | — | 6.75 | — | — | 6.75 |
| Probiotic ^{§} | — | — | 0.15 | — | 0.15 |
| Transfer factor | — | — | — | 0.26 | 0.26 |

Carbohydrate (g/kg)

| | | | | | |
|---|---|---|---|---|---|
| Cellulose | 30 | 30 | 30 | 30 | 30 |
| Corn starch | 230.0 | 230.0 | 230.0 | 230.0 | 230.0 |
| Maltodextrin ^{‡} | 70.0 | 63.2 | 69.7 | 69.6 | 62.7 |
| Sucrose | 261.3 | 261.3 | 261.3 | 261.3 | 261.3 |

Protein (g/kg)

| | | | | | |
|---|---|---|---|---|---|
| Casein | 190 | 190 | 190 | 190 | 190 |
| L-cysteine | 2.85 | 2.85 | 2.85 | 2.85 | 2.85 |

Fat (g/kg)

| | | | | | |
|---|---|---|---|---|---|
| Anhydrous milk fat | 36.3 | 36.3 | 36.3 | 36.3 | 36.3 |
| Beef tallow | 24.8 | 24.8 | 24.8 | 24.8 | 24.8 |
| Cholesterol | 0.4 | 0.4 | 0.4 | 0.4 | 0.4 |
| Corn Oil | 16.5 | 16.5 | 16.5 | 16.5 | 16.5 |
| Lard | 28 | 28 | 28 | 28 | 28 |
| Olive oil | 28 | 28 | 28 | 28 | 28 |
| Soybean oil | 31.4 | 31.4 | 31.4 | 31.4 | 31.4 |

Vitamin, mineral, antioxidant (g/kg)

| | | | | | |
|---|---|---|---|---|---|
| Mineral mix | 35 | 35 | 35 | 35 | 35 |
| Vitamin mix | 10 | 10 | 10 | 10 | 10 |
| Sodium chloride | 4 | 4 | 4 | 4 | 4 |
| Choline bitartrate | 1.4 | 1.4 | 1.4 | 1.4 | 1.4 |
| TBHQ | 0.028 | 0.028 | 0.028 | 0.028 | 0.028 |

% Kcal

| | | | | | |
|---|---|---|---|---|---|
| Protein | 15.5 | 15.5 | 15.5 | 15.5 | 15.5 |
| Carbohydrate | 50.0 | 49.7 | 50.0 | 50.0 | 49.7 |
| Fat | 34.5 | 34.7 | 34.5 | 34.5 | 34.7 |
| Calorie (Kcal/g) | 4.4 | 4.3 | 4.4 | 4.4 | 4.3 |

^{§} The prebiotics contained equal parts fructooligosaccharides, galactooligosaccharides, and xylooligosaccharides.
^{‡} Prebiotics, probiotics and Transfer Factor additions were balanced by removing maltodextrin.

### Study Design

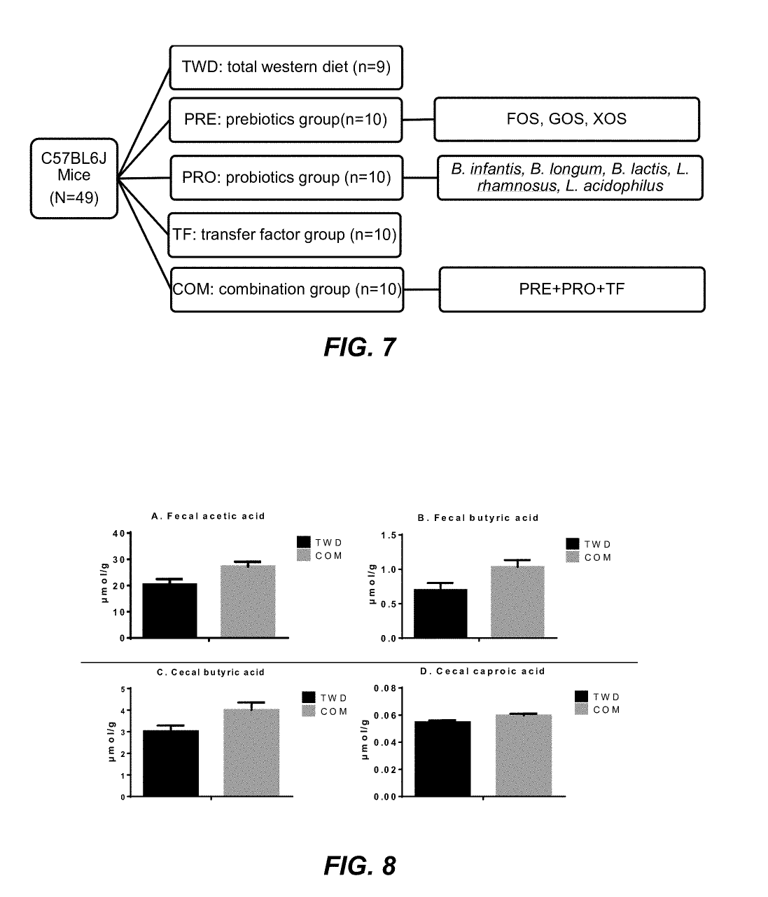

Healthy C57Bl/6J male mice were purchased from Jackson Laboratories (Bar Harbor, Me.). Mice were randomly assigned to each treatment for four (4) weeks. Mice were individually housed in HEPA-filtered micro isolator cages. A twelve (12) hour light/dark cycle was used, and the room temperature was kept between 18-23° C. with humidity between 20-50%. All animal care and husbandry procedures were performed under the Animal Welfare Act and the Public Health Service Policy on Humane Care and Use of Laboratory Animals, as well as USU Institutional Animal Care and Use Committee Protocol #2640. The experimental design is shown in FIG. 7.

Food intake and body weight were measured twice weekly. At the end of intervention, mice were killed by CO₂ asphyxiation. Blood was removed by cardiac puncture and plasma was separated from whole blood via centrifugation. Plasma was aliquoted into microcentrifuge tubes and snap frozen in liquid nitrogen. Both fecal and cecal samples were collected at the end of intervention and snap frozen in liquid nitrogen and stored at −80° C. until analysis.

### SCFAs Analysis

Short chain fatty acids (SCFAs), which are also referred to as volatile fatty acids, are fatty acids with less than six (6) carbon atoms. SCFAs are produced by bacteria when they ferment dietary components (primarily non-digestible carbohydrates, such as fiber) inside the colon. SCFAs were extracted from fecal and cecal samples at the end of intervention, and measured by gas chromatography with flame ionization detection (GC-FID) according to the method from Ward et al [41].

### Gut Microbiome

Fecal bacterial DNA (deoxyribonucleic acid) was extracted using a commercial extraction according to the manufacturer's instructions (QIAmp Fast DNA Stool mini Kit, Qiagen, Germantown, Md.). After extraction, DNA samples were normalized, and amplified via PCR (polymerase chain reaction) using barcoded primers directed against the V3 region of the 16S rRNA (ribosomal ribonucleic acid). PCR products were purified sequenced at the Utah State Center for Integrated Biotechnology core sequencing facility using the Ion PGM System and analyzed using Ion Reporter™ workflow. Microbiota sequences were processed through the QIIME version 1.9 [42]. After quality filtering and sample assignment, sequences were clustered into operational taxonomic units (OTUs) [43] at a 97% sequence similarity against a reference GreenGenes OTU database (gg_13_8_otus) using the open-reference OTU picking approach with UCLUST [44]. The most abundant sequence from each cluster were selected as representative sequences and checked for chimeras using uchime. Alpha and beta diversity analysis were performed using jackknifed_beta_diversity.py and alpha_diversity.py workflow scripts respectively.

### Gut Inflammation

Calprotectin is a protein that is released by white blood cells, specifically, neutrophils, as the neutrophils gather at inflamed locations of the gut. Fecal calprotectin was extracted by with the following extraction buffer: 0.1 M Tris, 0.15 M NaCl, 1.0 M urea, 10 mM CaCl2, 0.1 M citric acid monohydrate and 5 g/L BSA (bovine serum albumin) (pH 8.0). After extraction and centrifugation, the supernatant was used for the ELISA (enzyme-linked immunosorbent assay) analysis with a commercial kit following manufacturer's instructions (Hycult Inc., Wayne, Pa.).

### Plasma Zonulin

Zonulin is a protein that modulates permeability of the gut. More specifically, zonulin modulates the permeability of tight junctions between cells of the wall of the gut. Plasma samples were diluted and analyzed using a commercial ELISA kit according to manufacturer's directions (MyBioSource, San Diego, Calif.) to determine zonulin levels in the plasma.

### Diet Probiotic Enumeration

Diet samples were sent to Covance Laboratory (Madison, Wis.) for Total Probiotic Enumeration using standard procedures [45].

### Statistics Analysis

Treatment effects and interactions were determined by one way-ANOVA with Tukey HSD (honestly significant difference) post hoc test. In some cases, Student's T-Test was used to compare the TWD and COM treatments, as the probiotic composition contains prebiotics, probiotics, and an immune modulator. For all statistical tests, a p value <0.05 (two-tailed test) was considered as significant. Transformations were used to equalize variance prior to the statistical analyses in cases where variance assumptions were not met.

### Results

### Diet Probiotic Content

The probiotics were added to the PRO and COM diets as powders, and plate counts were conducted by a third party to enumerate the colony forming units (CFU) in each diet. These numbers were then used to determine the average probiotic intake for each diet (TABLE 6).

TABLE 6
Probiotic enumeration for diets, and estimated probiotic intake/d

| Treatment | TWD | PRE | PRO | TF | COM |
|---|---|---|---|---|---|
| CFU/g diet | <1 × 10⁴ | 2 × 10⁴ | 9 × 10⁴ | <1 × 10⁴ | 3.4 × 10⁵ |
| CFU/d^{‡} | <2.4 × 10⁴ | 5.5 × 10⁴ | 2.6 × 10⁵ | <2.7 × 10⁴ | 9 × 10⁶ |

^{‡}Probiotic intake was estimated using CFU/g content measured in diets and average mass of food consumed per group.

In the TWD and TF diets, the probiotic plate counts were below the detection limit of the assay, which is not surprising, as probiotics were not added to the diets. The PRE diet did contain a measurable level of probiotics, which may have been present in the prebiotic powders. The PRO diet contained a higher level of probiotics. The COM diet included the highest level of probiotics.

### Food Intake, Weight Gain, Metabolic Efficiency, and Probiotic Intake

Mice consumed significantly more calories on the PRO diet than the TWD (TABLE 7), but there were no other differences in intake among the diets. The treatments did not appear to have any effect on weight gain or metabolic efficiency, which is the increase in mass, or mass gain, per calorie.

TABLE 7
Food intake, weight gain, and metabolic efficiency

| Treatment | TWD | PRE | PRO | TF | COM |
|---|---|---|---|---|---|
| Energy intake (Kcal/day) | 10.8 ± 0.2^{a} | 12.1 ± 0.2^{ab} | 12.9 ± 0.5^{b} | 12.2 ± 0.4^{ab} | 11.7 ± 0.4^{ab} |
| Weight gain (g) | 6.6 ± 0.5 | 8.0 ± 0.5 | 7.7 ± 0.5 | 6.4 ± 0.4 | 7.4 ± 0.6 |
| Metabolic efficiency | 0.61 ± 0.04 | 0.66 ± 0.04 | 0.60 ± 0.04 | 0.53 ± 0.03 | 0.63 ± 0.05 |
| (g/kcal) | | | | | |

Values with different superscripts differed significantly (p < 0.05).

### SCFAs

There were very few differences in the SCFA content of the cecal or fecal samples (TABLE 8). In the cecal contents, only caproic acid differed significantly between the treatments, with all treatments being higher than the control. In feces, there was a trend (p<0.1) for differences in iso-butyric and valeric acids.

TABLE 8
SCFAs in fecal samples for treatments

| Cecal SCFAs | | | | | | p-value |
|---|---|---|---|---|---|---|
| (μmol/g) | TWD | PRE | PRO | TF | COM | (ANOVA) |
| Acetic acid | 27.8 ± 2.1 | 26.3 ± 2.5 | 26.4 ± 1.7 | 28.9 ± 2.1 | 28.3 ± 1.3 | 0.84 |
| Propionic acid | 3.87 ± 0.28 | 3.88 ± 0.29 | 4.58 ± 0.30 | 4.11 ± 0.28 | 4.61 ± 0.33 | 0.22 |
| n-Butyric acid | 3.02 ± 0.30 | 3.30 ± 0.43 | 3.38 ± 0.23 | 3.62 ± 0.35 | 4.00 ± 0.35 | 0.33 |
| iso-Butyric acid | 0.48 ± 0.01 | 0.44 ± 0.03 | 0.47 ± 0.01 | 0.44 ± 0.02 | 0.47 ± 0.01 | 0.47 |
| iso-Valeric acid | 0.53 ± 0.01 | 0.49 ± 0.02 | 0.51 ± 0.02 | 0.51 ± 0.03 | 0.52 ± 0.03 | 0.79 |
| n-Valeric acid | 0.51 ± 0.02 | 0.48 ± 0.04 | 0.49 ± 0.04 | 0.52 ± 0.05 | 0.52 ± 0.03 | 0.86 |
| Caproic acid | 0.05 ± 0.01 | 0.06 ± 0.01 | 0.06 ± 0.01 | 0.06 ± 0.01 | 0.06 ± 0.01 | 0.05 |
| Fecal SCFAs | | | | | | p-value |
| (μmol/g) | TWD | PRE | PRO | TF | COM | (ANOVA) |
| Acetic acid | 20.4 ± 2.1 | 28.0 ± 2.5 | 25.5 ± 4.5 | 22.8 ± 1.8 | 27.2 ± 1.9 | 0.27 |
| Propionic acid | 2.2 ± 0.3 | 2.6 ± 0.2 | 2.1 ± 0.3 | 2.6 ± 0.3 | 2.8 ± 0.2 | 0.20 |
| n-Butyric acid | 0.70 ± 0.10 | 0.97 ± 0.17 | 0.87 ± 0.08 | 1.05 ± 0.12 | 1.03 ± 0.10 | 0.26 |
| iso-Butyric acid | 0.26 ± 0.03 | 0.37 ± 0.04 | 0.25 ± 0.03 | 0.35 ± 0.05 | 0.32 ± 0.02 | 0.06 |
| iso-Valeric acid | 0.43 ± 0.05 | 0.58 ± 0.05 | 0.45 ± 0.03 | 0.55 ± 0.07 | 0.52 ± 0.02 | 0.21 |
| n-Valeric acid | 0.19 ± 0.03 | 0.26 ± 0.04 | 0.14 ± 0.02 | 0.31 ± 0.08 | 0.19 ± 0.02 | 0.10 |
| Caproic acid | 0.06 ± 0.01 | 0.17 ± 0.06 | 0.09 ± 0.02 | 0.20 ± 0.10 | 0.11 ± 0.03 | 0.39 |

SCFAs are expressed as mean ± SE (μmol/g).
P-value was calculated by one-way ANOVA.

FIG. 8 depicts the results of the SCFA analysis of the contents of cecal and fecal samples from the mice. When the TWD and COM treatments are compared directly, there was more butyric and caproic acid in the cecal contents, and more acetic and butyric acid in the fecal content.

### Microbiome—Taxonomic Summaries

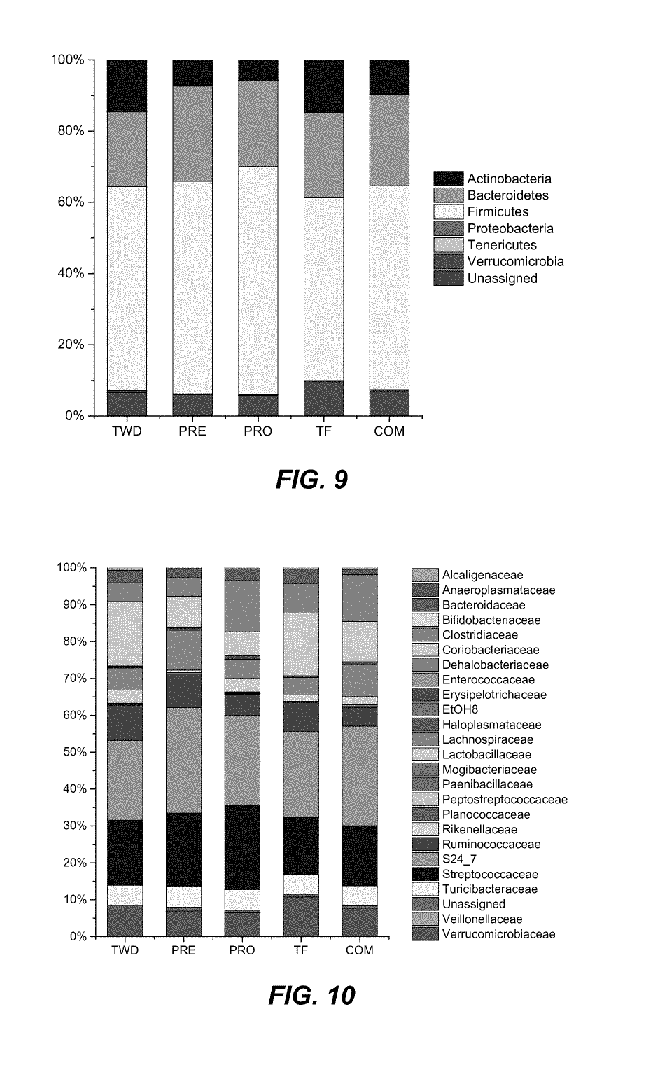

After quality, chimera, and abundance filtering, sequences were assigned to OTUs using the pick_open_ref_otus command for an average of 46853 sequences per sample assigned to 1546 OTUs. FIGS. 9 and 10 respectively show the microbiome composition at the phylum level and the family level. Firmicutes and Bacteroidetes are two dominant bacterial divisions in phylum taxonomy. There was no significant statistical difference in phylum level taxonomy and Firmicutes:Bacteroidetes ratio. But the treatments affected the microbiome composition in family and genus level taxonomy. A summary of significant differences in relative abundance is provided by in TABLE 9.

TABLE 9
Significant Effects of Diet on Taxonomic Abundance in Mice

| | | |
|---|---|---|
| | P-Value | |
| Taxa | (FDR corrected) | Direction* |

Class

| | | |
|---|---|---|
| Actinobacteria | <0.001 | Higher in PRO and COM |

Order

| | | |
|---|---|---|
| Bifidobacteriales | <0.001 | Higher in PRO and COM |

Family

| | | |
|---|---|---|
| Bifidobacteriaceae | <0.001 | Higher in PRO and COM |
| Lachnospiraceae | 0.008 | Higher in PRE and COM |
| Ruminococcaceae | 0.044 | Lower in PRO and COM |

Genus

| | | |
|---|---|---|
| Bifidobacterium | <0.001 | Higher in PRO and COM |
| Ruminococcus | <0.001 | Higher in PRE and COM |
| Oribacterium | 0.049 | Higher in PRE and COM |

Species

| | | |
|---|---|---|
| B. longum | <0.001 | Higher in PRO and COM |
| R. gnavus | <0.001 | Higher in PRE and COM |

*represent the different groups in analysis

At the family level, Bifidobacteriaceae abundance was significantly increased in PRO and COM. Lachnospiraceae abundance was significantly increased in PRE and COM. Ruminococcaceae was significantly decreased in PRO and COM. At the genus level, Bifidobacterium abundance was significantly increased in PRO and COM. The abundance of Ruminococcus and Oribacterium were significantly increased in PRE and COM.

### Microbiome Diversity

Alpha-diversity and beta-diversity may be used to evaluate the variation of microbiome composition. The diversity analysis may provide an understanding of similarity, replacement, and richness difference within site and among sites [61]. Gut microbiome diversity has been negatively associated with weight gain, while it has been positively correlated with fiber intake [62]. Patients with inflammatory bowel diseases (IBD) typically have a lower diversity in gut bacteria, with a reduction of the dominant Firmicutes and Bacteroidetes, when compared with healthy people [63, 64].

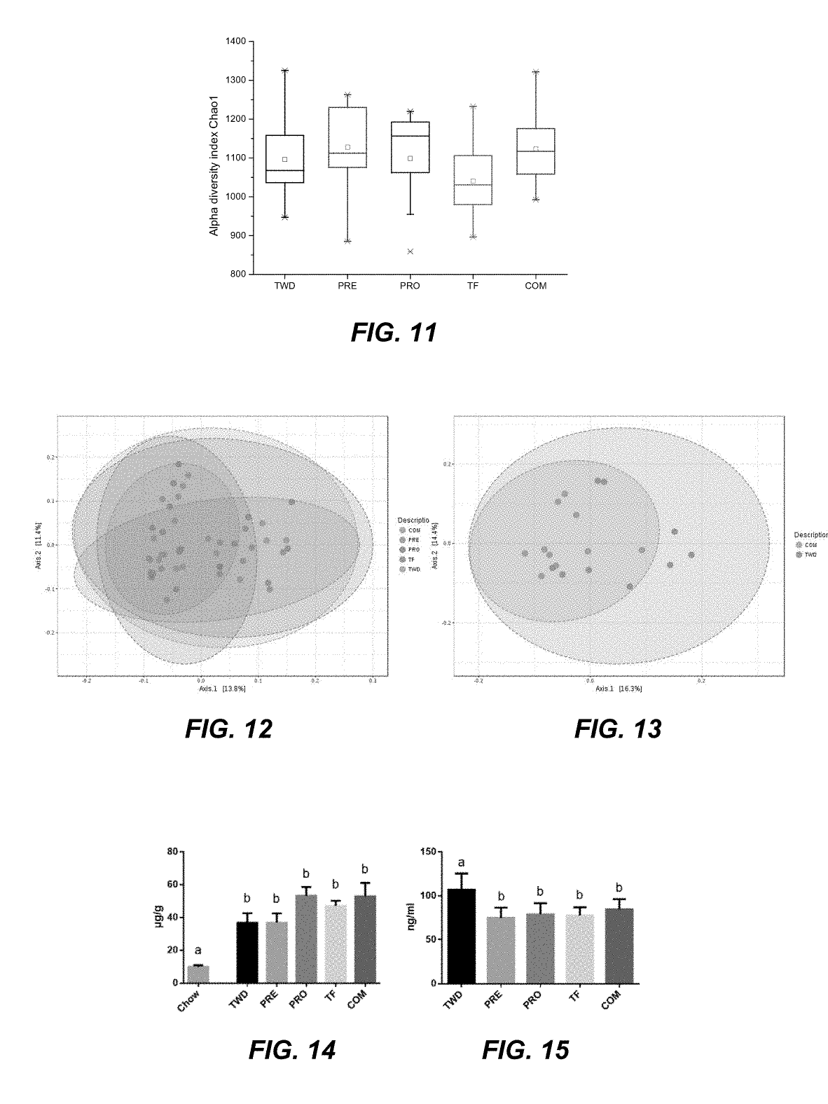

Alpha diversity refers to within-habitat diversity. It is the component of total diversity that can be attributed to the average number of species found within homogeneous sampling units (i.e., habitats) [46]. Alpha diversity was determined using Chao1 index. The analyses showed that no significant difference affected by diets, as depicted by FIG. 11.

Beta diversity refers to between-habitat diversity. It is the component of total diversity that can be attributed to differences in species composition among the homogeneous units in the landscape [46]. FIG. 12 is a spatial representation of beta diversity with Principal Coordinates Analysis (PCoA). There was a significant effect of overall treatment on beta diversity (p<0.002) using unweighted UniFrac distance with a non-parametric PERMANOVA test. The trends in beta diversity in the TWD and TF groups were similar to each other, but different from the beta diversity trends in other groups. Significant beta diversity was observed in the COM group relative to the TWD group (p<0.005), as depicted by FIG. 13.

The dosages that were used in the study were based more closely on the dosages that humans would receive, as opposed to the megadoses that are typically administered to mice. While no statistically significant difference in alpha diversity was detected, the treatments did increase beta diversity.

### Gut Inflammation

The effects of the various diets on fecal calprotectin is shown in FIG. 14. Prior to being randomized to the treatments, fecal samples were collected from mice consuming a standard laboratory chow diet. According to the data shown in FIG. 14, mice consuming chow had lower levels of fecal calprotectin than mice on any of the treatment diets. There was no statistically significant treatment effect among the various test diets (p=0.1355).

### Gut Permeability

As shown in FIG. 15, plasma zonulin was lower in mice fed the PRE, PRO, TF, and COM treatments than in the mice of the control (TWD) group (p=0.0006). The extent to which zonulin levels increased in the COM group suggests that the COM treatment may improve modulation of the barrier function of the gut, which may prevent potentially harmful substances (e.g., pathogens, etc.) from passing through the wall of the gut and further into a subject's body.

Although the foregoing description provides many specifics, these should not be construed as limiting the scope of any of the appended claims, but merely as providing illustrations of some embodiments of the disclosed subject matter. Similarly, other embodiments may be devised. Features from different embodiments may be employed in combination. The scope of each claim should, therefore, be indicated and limited only by the appended claims and their legal equivalents. All additions, deletions, and modifications to the disclosed subject matter that fall within the meanings and scopes of the claims are to be embraced by the claims.

### REFERENCES

1. Gibson, G. R., et al., Expert consensus document: The International Scientific Association for Probiotics and Prebiotics (ISAPP) consensus statement on the definition and scope of prebiotics. Nature Reviews Gastroenterology and Hepatology, 2017. 14(8): p. 491.
2. Gibson, G. R., et al., Dietary modulation of the human colonic microbiota: updating the concept of prebiotics. Nutrition research reviews, 2004. 17(2): p. 259-275.
3. Hill, C., et al., Expert consensus document. The International Scientific Association for Probiotics and Prebiotics consensus statement on the scope and appropriate use of the term probiotic. Nat Rev Gastroenterol Hepatol, 2014. 11(8): p. 506-14.
4. Hill, C., et al., Expert consensus document: The International Scientific Association for Probiotics and Prebiotics consensus statement on the scope and appropriate use of the term probiotic. Nature Reviews Gastroenterology and Hepatology, 2014. 11(8): p. 506.
5. Mack, D. R., Probiotics: Mixed Messages. Canadian Family Physician, 2005. 51(11): p. 1455.
6. Roberfroid, M., Prebiotics: the concept revisited. The Journal of nutrition, 2007. 137(3): p. 830S-837S.
7. Blaut, M., Relationship of prebiotics and food to intestinal microflora. Eur J Nutr, 2002. 41 Suppl 1: p. I11-6.
8. Rastall, R. A., Functional oligosaccharides: application and manufacture. Annu Rev Food Sci Technol, 2010. 1: p. 305-39.
9. Swennen, K., C. M. Courtin, and J. A. Delcour, Non-digestible oligosaccharides with prebiotic properties. Crit Rev Food Sci Nutr, 2006. 46(6): p. 459-71.
10. Cho, I. and M. J. Blaser, The human microbiome: at the interface of health and disease. Nature Reviews Genetics, 2012. 13(4): p. nrg3182.
11. Saez-Lara, M. J., et al., Effects of Probiotics and Synbiotics on Obesity, Insulin Resistance Syndrome, Type 2 Diabetes and Non-Alcoholic Fatty Liver Disease: A Review of Human Clinical Trials. Int J Mol Sci, 2016. 17(6).
12. Delbes, A. S., et al., Prebiotics Supplementation Impact on the Reinforcing and Motivational Aspect of Feeding. Front Endocrinol (Lausanne), 2018. 9: p. 273.
13. Nihei, N., et al., Dietary alpha-cyclodextrin modifies gut microbiota and reduces fat accumulation in high-fat-diet-fed obese mice. Biofactors, 2018.
14. Cheng, W., et al., Effect of Functional Oligosaccharides and Ordinary Dietary Fiber on Intestinal Microbiota Diversity. Front Microbiol, 2017. 8: p. 1750.
15. Hamilton, M. K., et al., Prebiotic milk oligosaccharides prevent development of obese phenotype, impairment of gut permeability, and microbial dysbiosis in high fat-fed mice. Am J Physiol Gastrointest Liver Physiol, 2017. 312(5): p. G474-G487.
16. Vandeputte, D., et al., Prebiotic inulin-type fructans induce specific changes in the human gut microbiota. Gut, 2017. 66(11): p. 1968-1974.
17. Clarke, S. T., et al., beta2-1 Fructan supplementation alters host immune responses in a manner consistent with increased exposure to microbial components: results from a double-blinded, randomised, cross-over study in healthy adults. Br J Nutr, 2016. 115(10): p. 1748-59.
18. Holscher, H. D., et al., Agave Inulin Supplementation Affects the Fecal Microbiota of Healthy Adults Participating in a Randomized, Double-Blind, Placebo-Controlled, Crossover Trial. J Nutr, 2015. 145(9): p. 2025-32.
19. Finegold, S. M., et al., Xylooligosaccharide increases bifidobacteria but not lactobacilli in human gut microbiota. Food Funct, 2014. 5(3): p. 436-45.
20. Lecerf, J. M., et al., Xylo-oligosaccharide (XOS) in combination with inulin modulates both the intestinal environment and immune status in healthy subjects, while XOS alone only shows prebiotic properties. Br J Nutr, 2012. 108(10): p. 1847-58.
21. Wilms, E., et al., Effects of Supplementation of the Synbiotic Ecologic(R) 825/FOS P6 on Intestinal Barrier Function in Healthy Humans: A Randomized Controlled Trial. PLOS ONE, 2016. 11(12): p. e0167775.
22. Rajkumar, H., et al., Effect of Probiotic Lactobacillus salivarius UBL S22 and Prebiotic Fructo-oligosaccharide on Serum Lipids, Inflammatory Markers, Insulin Sensitivity, and Gut Bacteria in Healthy Young Volunteers: A Randomized Controlled Single-Blind Pilot Study. J Cardiovasc Pharmacol Ther, 2015. 20(3): p. 289-98.
23. Childs, C. E., et al., Xylo-oligosaccharides alone or in synbiotic combination with Bifidobacterium animalis subsp. lactis induce bifidogenesis and modulate markers of immune function in healthy adults: a double-blind, placebo-controlled, randomised, factorial cross-over study. Br J Nutr, 2014. 111(11): p. 1945-56.
24. Rucker, R. B., Allometric scaling, metabolic body size and interspecies comparisons of basal nutritional requirements. Journal of animal physiology and animal nutrition, 2007. 91(3-4): p. 148-56.
25. Carasi, P., et al., Impact of kefir derived Lactobacillus kefiri on the mucosal immune response and gut microbiota. J Immunol Res, 2015. 2015: p. 361604.
26. Mariman, R., et al., The Probiotic Mixture VSL#3 Has Differential Effects on Intestinal Immune Parameters in Healthy Female BALB/c and C57BL/6 Mice. J Nutr, 2015. 145(6): p. 1354-61.
27. Bal, Z., et al., Impact of Environmental Microbes on the Composition of the Gut Microbiota of Adult BALB/c Mice. PLOS ONE, 2016. 11(8): p. e0160568.
28. Wang, S., et al., Fermented milk supplemented with probiotics and prebiotics can effectively alter the intestinal microbiota and immunity of host animals. J Dairy Sci, 2012. 95(9): p. 4813-4822.
29. Seo, M., et al., Methanobrevibacter attenuation via probiotic intervention reduces flatulence in adult human: A non-randomised paired-design clinical trial of efficacy. PLOS ONE, 2017. 12(9): p. e0184547.
30. Rungsri, P., et al., Effect of fermented milk containing Lactobacillus rhamnosus SD11 on oral microbiota of healthy volunteers: A randomized clinical trial. J Dairy Sci, 2017. 100(10): p. 7780-7787.
31. Lee, Y., et al., Effects of Bifidobacterium animalis subsp. lactis BB-12((R)) on the lipid/lipoprotein profile and short chain fatty acids in healthy young adults: a randomized controlled trial. Nutr J, 2017. 16(1): p. 39.
32. Toscano, M., et al., Effect of Lactobacillus rhamnosus HN001 and Bifidobacterium longum BB536 on the healthy gut microbiota composition at phyla and species level: A preliminary study. World J Gastroenterol, 2017. 23(15): p. 2696-2704.
33. Gargari, G., et al., Consumption of a Bifidobacterium bifidum Strain for 4 Weeks Modulates Dominant Intestinal Bacterial Taxa and Fecal Butyrate in Healthy Adults. Appl Environ Microbiol, 2016. 82(19): p. 5850-9.
34. van Zanten, G. C., et al., Synbiotic Lactobacillus acidophilus NCFM and cellobiose does not affect human gut bacterial diversity but increases abundance of lactobacilli, bifidobacteria and branched-chain fatty acids: a randomized, double-blinded cross-over trial. FEMS Microbiol Ecol, 2014. 90(1): p. 225-36.
35. Foure, M., et al., Chicory Roots for Prebiotics and Appetite Regulation: A Pilot Study in Mice. J Agric Food Chem, 2018. 66(25): p. 6439-6449.
36. Singh, D. P., et al., Isomalto-oligosaccharides, a prebiotic, functionally augment green tea effects against high fat diet-induced metabolic alterations via preventing gut dysbacteriosis in mice. Pharmacol Res, 2017. 123: p. 103-113.
37. Umu, O. C., et al., The Potential of Class II Bacteriocins to Modify Gut Microbiota to Improve Host Health. PLOS ONE, 2016. 11(10): p. e0164036.
38. Hintze, K. J., et al., Modeling the Western Diet for Preclinical Investigations. Advances in Nutrition, 2018: p. nmy002-nmy002.
39. Fernandes, R., et al., Effects of inulin-type fructans, galacto-oligosaccharides and related synbiotics on inflammatory markers in adult patients with overweight or obesity: A systematic review. Clinical nutrition, 2017. 36(5): p. 1197-1206.
40. Beserra, B. T., et al., A systematic review and meta-analysis of the prebiotics and synbiotics effects on glycaemia, insulin concentrations and lipid parameters in adult patients with overweight or obesity. Clinical nutrition, 2015. 34(5): p. 845-58.
41. Ward, R. E., et al., Consumption of the total Western diet differentially affects the response to green tea in rodent models of chronic disease compared to the AIN93G diet. Mol Nutr Food Res, 2016.
42. Caporaso, J. G., et al., QIIME allows analysis of high-throughput community sequencing data. Nat Methods, 2010. 7(5): p. 335-6.
43. Schloss, P. D., et al., Introducing mothur: open-source, platform-independent, community-supported software for describing and comparing microbial communities. Appl Environ Microbiol, 2009. 75(23): p. 7537-41.
44. DeSantis, T. Z., et al., Greengenes, a chimera-checked 16S rRNA gene database and workbench compatible with ARB. Appl Environ Microbiol, 2006. 72(7): p. 5069-72.
45. Schoeni, J. L., 20. Probiotics, in Compendium of Methods for the Microbiological Examination of Foods 2013, American Public Health Association.
46. Gering, J. C. and T. O. Crist, The alpha-beta-regional relationship: providing new insights into local-regional patterns of species richness and scale dependence of diversity components. Ecology Letters, 2002. 5(3): p. 433-444.
47. Hamilton, B. S., et al., A library of chemically defined human N-glycans synthesized from microbial oligosaccharide precursors. Scientific reports, 2017. 7(1): p. 15907.
48. Weitkunat, K., et al., Effects of dietary inulin on bacterial growth, short-chain fatty acid production and hepatic lipid metabolism in gnotobiotic mice. The Journal of nutritional biochemistry, 2015. 26(9): p. 929-37.
49. Murakami, Y., et al., Supplemental epilactose prevents metabolic disorders through uncoupling protein-1 induction in the skeletal muscle of mice fed high-fat diets. Br J Nutr, 2015. 114(11): p. 1774-83.
50. Murakami, Y., et al., Supplemental epilactose prevents metabolic disorders through uncoupling protein-1 induction in the skeletal muscle of mice fed high-fat diets. The British journal of nutrition, 2015. 114(11): p. 1774-83.
51. Weitkunat, K., et al., Effects of dietary inulin on bacterial growth, short-chain fatty acid production and hepatic lipid metabolism in gnotobiotic mice. J Nutr Biochem, 2015. 26(9): p. 929-37.
52. Institute_of_Medicine, Dietary Reference Intakes for Energy, Carbohydrate, Fiber, Fat, Fatty Acids, Cholesterol, Protein, and Amino Acids 2005, Washington, D.C.: The National Academies Press. 1358.
53. Johnson-Henry, K. C., et al., Short-chain fructo-oligosaccharide and inulin modulate inflammatory responses and microbial communities in Caco2-bbe cells and in a mouse model of intestinal injury. J Nutr, 2014. 144(11): p. 1725-33.
54. Everard, A., et al., Microbiome of prebiotic-treated mice reveals novel targets involved in host response during obesity. ISME J, 2014. 8(10): p. 2116-30.
55. Unit, M. C., et al., Rapidly expanding knowledge on the role of the gut microbiome in health and disease. Biochimica et Biophysica Acta (BBA)—Molecular Basis of Disease, 2014. 1842(10): p. 1981-1992.
56. Cheng, W., et al., Effects of a galacto-oligosaccharide-rich diet on fecal microbiota and metabolite profiles in mice. Food & function, 2018. 9(3): p. 1612-1620.
57. Frece, J., et al., Synbiotic effect of Lactobacillus helveticus M92 and prebiotics on the intestinal microflora and immune system of mice. J Dairy Res, 2009. 76(1): p. 98-104.
58. Cortez-Pinto, H., et al., Microbiota Modulation With Synbiotic Decreases Liver Fibrosis in a High Fat Choline Deficient Diet Mice Model of Non-Alcoholic Steatohepatitis (NASH). GE Port J Gastroenterol, 2016. 23(3): p. 132-141.
59. Mischke, M., et al., Specific synbiotics in early life protect against diet-induced obesity in adult mice. Diabetes Obes Metab, 2018. 20(6): p. 1408-1418.
60. Turnbaugh, P. J., et al., A core gut microbiome in obese and lean twins. Nature, 2008. 457: p. 480.
61. Legendre, P., Interpreting the replacement and richness difference components of beta diversity. Global Ecology and Biogeography, 2014. 23(11): p. 1324-1334.
62. Menni, C., et al., Gut microbiome diversity and high-fibre intake are related to lower long-term weight gain. Int J Obes (Lond), 2017. 41(7): p. 1099-1105.
63. Manichanh, C., et al., Reduced diversity of faecal microbiota in Crohn's disease revealed by a metagenomic approach. Gut, 2006. 55(2): p. 205-211.
64. Sokol, H., et al., Analysis of bacterial bowel communities of IBD patients: What has it revealed? Inflammatory bowel diseases, 2008. 14(6): p. 858-867.
65. Park, D. Y., et al., Supplementation of Lactobacillus curvatus HY7601 and Lactobacillus plantarum KY1032 in diet-induced obese mice is associated with gut microbial changes and reduction in obesity. PLOS ONE, 2013. 8(3): p. e59470.
66. Fallahi, G., et al., The effect of probiotics on fecal calprotectin in patients with cystic fibrosis. Turkish Journal of Pediatrics, 2013. 55(5): p. 475-478.
67. Bunn, S. K., et al., Fecal calprotectin: validation as a noninvasive measure of bowel inflammation in childhood inflammatory bowel disease. Journal of pediatric gastroenterology and nutrition, 2001. 33(1): p. 14-22.
68. Urban, C. F., et al., Neutrophil extracellular traps contain calprotectin, a cytosolic protein complex involved in host defense against Candida albicans. PLoS Pathog, 2009. 5(10): p. e1000639.
69. Konikoff, M. R. and L. A. Denson, Role of fecal calprotectin as a biomarker of intestinal inflammation in inflammatory bowel disease. Inflammatory bowel diseases, 2006. 12(6): p. 524-534.
70. Garcia, G., et al., Gut-borne Saccharomyces cerevisiae, a promising candidate for the formulation of feed additives, modulates immune system and gut microbiota. Beneficial microbes, 2016. 7(5): p. 659-668.

What is claimed:

1. A composition comprising:
an immune modulating component; and
a probiotic component.

2. The composition of claim 1, wherein the immune modulating component comprises a fraction of at least one of colostrum and eggs lacking transfer factor, the fraction capable of moderating metabolic activity of T-cells.

3. The composition of claim 2, wherein the fraction comprises bovine colostrum, egg yolk, and bovine colostrum extract.

4. The composition of claim 3, wherein the fraction has an upper molecular weight cutoff of 3,000 Da.

5. The composition of claim 3, wherein the immune modulating component comprises a weight ratio of about 68:30:2 of bovine colostrum:egg yolk:bovine colostrum extract.

6. The composition of claim 5, wherein the one or more prebiotics of the immune modulating component comprises one or more of 3′-sialyllactose, 6′-sialyllactose, and 6′ sialyl-N-acetyllactosamine.

7. The composition of claim 1, further comprises a prebiotic component.

8. The composition of claim 6, wherein the prebiotic component comprises at least one of xylooligosaccharides, galactooligosaccharides, and fructooligosaccharides.

9. The composition of claim 8, wherein the prebiotic component has a weight ratio of about 2:1:2 of xylooligosaccharides to galactooligosaccharides to fructooligosaccharides.

10. The composition of claim 1, wherein the probiotic component comprises a probiotic contained within a microcapsule.

11. The composition of claim 7, wherein the composition further comprises additives, and wherein the composition comprises about 8 percent by weight of the probiotic component, about 80 percent by weight of the prebiotic component, about 3 percent by weight of the immune modulating component, and about 9 percent by weight of the additives.

12. A composition consisting essentially of:
an immune modulating component;
a prebiotic component; and
a probiotic component.

13. The composition of claim 11, wherein the immune modulating component comprises at least one of bovine colostrum, egg yolk, and bovine colostrum extract.

14. The composition of claim 3, wherein the prebiotic component comprises at least one of one of xylooligosaccharides, galactooligosaccharides, and fructooligosaccharides.

15. The composition of claim 1, wherein the immune modulating component is capable of increasing growth of one or more probiotic bacterium.

16. A method for improving digestive health, the method comprising:
administering a compound to an animal, the compound comprising an immune modulating component, and a probiotic component.

17. The method of claim 16, wherein the compound further comprises a prebiotic component.

18. The method of claim 17, wherein the step of administering the compound to the animal comprises administering a daily dose, and wherein the daily dose comprises about 250 milligrams of the probiotic component, about 2,500 milligrams of the prebiotic component, and about 100 milligrams of the immune modulating component.

19. The method of claim 18, wherein the step further comprises administering the daily dose each day for thirty days.

20. The method of claim 19, wherein the step further comprises administering the daily dose every other day after the thirty days.
